(19) United States
(12) Patent Application Publication
(10) Pub. No.: US 2022/0133178 A1
(43) Pub. Date: May 5, 2022

(54) FIBROUS COVER LAYER FOR MEDICAL DEVICES

(71) Applicant: Cardiac Pacemakers, Inc., St. Paul, MN (US)

(72) Inventors: Yingbo Li, Woodbury, MN (US); Michael J. Kane, St. Paul, MN (US); David Robert Wulfman, Minneapolis, MN (US); Adeniyi O. Aremu, Ramsey, MN (US)

(21) Appl. No.: 17/510,802

(22) Filed: Oct. 26, 2021

Related U.S. Application Data
(60) Provisional application No. 63/107,349, filed on Oct. 29, 2020.

(51) Int. Cl. A61B 5/1473; A61B 5/145; A61B 5/1459

(52) U.S. Cl. CPC A61B 5/1473; A61B 5/14546; A61B 2562/12; A61B 2562/162; A61B 5/1459

(57) ABSTRACT

Embodiments herein relate to implantable medical devices including a fibrous cover layer. In an embodiment, an implantable medical device is included having a housing, an optical chemical sensing element disposed along the housing, and a fibrous electrospun cover layer, wherein the fibrous electrospun cover layer is disposed over the optical chemical sensing element. In another embodiment, a method of making an implantable medical device is included. The method can specifically include depositing an optical chemical sensing element into a sensor optical carrier attached to a housing and applying a fibrous electrospun cover layer over the optical chemical sensing element. Other embodiments are also included herein.

This application claims the benefit of U.S. Provisional Application No. 63/107,349, filed Oct. 29, 2020, the content of which is herein incorporated by reference in its entirety.

### FIELD

Embodiments herein relate to implantable medical devices and, more specifically, to implantable medical devices including a fibrous cover layer.

### BACKGROUND

Data regarding physiological analytes are highly relevant for the diagnosis and treatment of many conditions and disease states. As one example, potassium ion concentrations can affect a patient's cardiac rhythm. Therefore, medical professionals frequently evaluate physiological potassium ion concentration when diagnosing a cardiac rhythm problem. However, measuring physiological concentrations of analytes, such as potassium, generally requires drawing blood from the patient. Blood draws are commonly done at a medical clinic or hospital and therefore generally require the patient to physically visit a medical facility. As a result, despite their significance, physiological analyte concentrations are frequently measured only sporadically.

Implantable chemical sensors can be used to gather data about physiological analytes while a patient is away from a medical care facility and without needing to draw blood or another fluid from the patient. However, the design and construction of effective chemical sensors is subject to many challenges.

### SUMMARY

Embodiments herein relate to implantable medical devices including a fibrous cover layer. In a first aspect, an implantable medical device is included having a housing, an optical chemical sensing element disposed along the housing, and a fibrous electrospun cover layer, wherein the fibrous electrospun cover layer is disposed over the optical chemical sensing element.

In a second aspect, in addition to one or more of the preceding or following aspects, or in the alternative to some aspects, the fibrous electrospun cover layer is from 10 microns to 2 millimeters thick.

In a third aspect, in addition to one or more of the preceding or following aspects, or in the alternative to some aspects, the housing is formed from a biostable metal.

In a fourth aspect, in addition to one or more of the preceding or following aspects, or in the alternative to some aspects, the housing is formed from titanium.

In a fifth aspect, in addition to one or more of the preceding or following aspects, or in the alternative to some aspects, the fibrous electrospun cover layer is disposed over the optical chemical sensing element and the housing.

In a sixth aspect, in addition to one or more of the preceding or following aspects, or in the alternative to some aspects, the fibrous electrospun cover layer encapsulates the housing.

In a seventh aspect, in addition to one or more of the preceding or following aspects, or in the alternative to some aspects, the fibrous electrospun cover layer is permeable to one or more physiological chemical elements.

In an eighth aspect, in addition to one or more of the preceding or following aspects, or in the alternative to some aspects, the fibrous electrospun cover layer is permeable to potassium and sodium.

In a ninth aspect, in addition to one or more of the preceding or following aspects, or in the alternative to some aspects, the fibrous electrospun cover layer is biocompatible.

In a tenth aspect, in addition to one or more of the preceding or following aspects, or in the alternative to some aspects, the fibrous electrospun cover layer can include polyethylene glycol.

In an eleventh aspect, in addition to one or more of the preceding or following aspects, or in the alternative to some aspects, the fibrous electrospun cover layer can include a copolymer can include polyethylene glycol subunits.

In a twelfth aspect, in addition to one or more of the preceding or following aspects, or in the alternative to some aspects, the fibrous electrospun cover layer can include thermoplastic fibers.

In a thirteenth aspect, in addition to one or more of the preceding or following aspects, or in the alternative to some aspects, the thermoplastic fibers have a diameter of 500 nanometers to 1 micron.

In a fourteenth aspect, in addition to one or more of the preceding or following aspects, or in the alternative to some aspects, the fibrous electrospun cover layer can include polyethylene glycol molecules, wherein the polyethylene glycol molecules are covalently bonded to the thermoplastic fibers.

In a fifteenth aspect, in addition to one or more of the preceding or following aspects, or in the alternative to some aspects, the fibrous electrospun cover layer can include a plurality of zones.

In a sixteenth aspect, in addition to one or more of the preceding or following aspects, or in the alternative to some aspects, wherein at least two of the plurality of zones have a different thickness, fiber density, fiber size, or fiber composition from one another.

In a seventeenth aspect, in addition to one or more of the preceding or following aspects, or in the alternative to some aspects, wherein at least one of the plurality of zones is disposed over the optical chemical sensing element and at least one of the plurality of zones is disposed over the housing.

In an eighteenth aspect, in addition to one or more of the preceding or following aspects, or in the alternative to some aspects, the implantable medical device can further include a sensor optical carrier attached to the housing.

In a nineteenth aspect, in addition to one or more of the preceding or following aspects, or in the alternative to some aspects, the optical chemical sensing element is disposed within the sensor optical carrier.

In a twentieth aspect, in addition to one or more of the preceding or following aspects, or in the alternative to some aspects, a top of the sensor optical carrier is flush with a surface of the housing.

In a twenty-first aspect, in addition to one or more of the preceding or following aspects, or in the alternative to some aspects, the implantable medical device can include a frame, wherein the frame is disposed between the housing and the fibrous electrospun cover layer.

In a twenty-second aspect, a method of making an implantable medical device is included, the method including applying a fibrous electrospun cover layer over a frame member, and fitting the frame member over at least one of a housing and a header of the implantable medical device.

In a twenty-third aspect, in addition to one or more of the preceding or following aspects, or in the alternative to some aspects, the fibrous electrospun cover layer is from 10 microns to 2 millimeters thick.

In a twenty-fourth aspect, in addition to one or more of the preceding or following aspects, or in the alternative to some aspects, the fibrous electrospun cover layer includes thermoplastic fibers.

In a twenty-fifth aspect, in addition to one or more of the preceding or following aspects, or in the alternative to some aspects, the thermoplastic fibers include polyethylene glycol.

In a twenty-sixth aspect, in addition to one or more of the preceding or following aspects, or in the alternative to some aspects, the fibrous electrospun cover layer is biocompatible.

In a twenty-seventh aspect, in addition to one or more of the preceding or following aspects, or in the alternative to some aspects, the fibrous electrospun cover layer is permeable to potassium and sodium.

In a twenty-eighth aspect, in addition to one or more of the preceding or following aspects, or in the alternative to some aspects, the fibrous electrospun cover layer is permeable to one or more physiological chemical elements.

In a twenty-ninth aspect, in addition to one or more of the preceding or following aspects, or in the alternative to some aspects, the fibrous electrospun cover layer includes a plurality of zones.

In a thirtieth aspect, a method of making an implantable medical device is included, the method including depositing an optical chemical sensing element into a sensor optical carrier attached to a housing, and applying a fibrous electrospun cover layer over the optical chemical sensing element.

In a thirty-first aspect, in addition to one or more of the preceding or following aspects, or in the alternative to some aspects, the method can further include applying the fibrous electrospun cover layer over the optical chemical sensing element and the housing.

In a thirty-second aspect, in addition to one or more of the preceding or following aspects, or in the alternative to some aspects, the fibrous electrospun cover layer encapsulates the housing.

In a thirty-third aspect, in addition to one or more of the preceding or following aspects, or in the alternative to some aspects, the fibrous electrospun cover layer is from 10 microns to 2 millimeters thick.

In a thirty-fourth aspect, in addition to one or more of the preceding or following aspects, or in the alternative to some aspects, the fibrous electrospun cover layer includes thermoplastic fibers.

In a thirty-fifth aspect, in addition to one or more of the preceding or following aspects, or in the alternative to some aspects, the thermoplastic fibers include polyethylene glycol.

In a thirty-sixth aspect, in addition to one or more of the preceding or following aspects, or in the alternative to some aspects, the fibrous electrospun cover layer is biocompatible.

In a thirty-seventh aspect, in addition to one or more of the preceding or following aspects, or in the alternative to some aspects, the fibrous electrospun cover layer is permeable to potassium and sodium.

In a thirty-eighth aspect, in addition to one or more of the preceding or following aspects, or in the alternative to some aspects, the fibrous electrospun cover layer is permeable to one or more physiological chemical elements.

In a thirty-ninth aspect, in addition to one or more of the preceding or following aspects, or in the alternative to some aspects, wherein applying a fibrous electrospun cover layer over the optical chemical sensing element further includes a plurality of zones.

In a fortieth aspect, in addition to one or more of the preceding or following aspects, or in the alternative to some aspects, wherein at least two of the plurality of zones have a different thickness, fiber density, fiber size, or fiber composition from one another.

In a forty-first aspect, in addition to one or more of the preceding or following aspects, or in the alternative to some aspects, wherein at least one of the plurality of zones is disposed over the optical chemical sensing element and at least one of the plurality of zones is disposed over the housing.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic top view of an implantable medical device in accordance with various embodiments herein.

FIG. 2 is a schematic view of an electrospinning process in accordance with various embodiments herein.

FIG. 3 is a schematic view of an implantable medical device coated with a fibrous layer in accordance with various embodiments herein.

FIG. 4 is a cross-sectional view of an implantable medical device coated with a fibrous layer as taken along line 4-4′ of FIG. 3.

FIG. 5 is a schematic view of an implantable medical device coated with a fibrous layer in accordance with various embodiments herein.

FIG. 6 is a cross-sectional view of an implantable medical device coated with a fibrous layer as taken along line 6-6′ of FIG. 5.

FIG. 7 is a schematic view of an implantable medical device coated with a fibrous layer in accordance with various embodiments herein.

FIG. 8 is a cross-sectional view of an implantable medical device coated with a fibrous layer as taken along line 8-8′ of FIG. 7.

FIG. 9 is a schematic view of an implantable medical device coated with a fibrous layer in accordance with various embodiments herein.

FIG. 10 is a cross-sectional view of an implantable medical device coated with a fibrous layer as taken along line 10-10′ of FIG. 9.

FIG. 11 is a flow diagram of a method of making an implantable medical device in accordance with various embodiments herein.

FIG. 12 is a flow diagram of a method of making an implantable medical device in accordance with various embodiments herein.

FIG. 13 is a schematic view of an implantable medical device in accordance with various embodiments herein.

FIG. 14 is a schematic diagram of components of an implantable medical device in accordance with various embodiments herein.

FIG. 15 is a schematic view of a fibrous layer being sprayed onto a frame in accordance with various embodiments herein.

FIG. 16 is a schematic view of placing a frame coated with a fibrous layer onto an implantable medical device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Physicians frequently analyze a patient's chemical analyte concentrations when assessing a patient's condition and/or diagnosing a patient's medical problem. For example, electrolyte concentrations (specifically including potassium) are commonly measured and analyzed by physicians due their impact on many bodily systems including the heart. Traditionally, chemical analyte concentrations are assessed by drawing a fluid sample, such as blood, from the patient while at a medical clinic or hospital. However, then a patient's chemical analyte concentrations can only be monitored when a patient physically visits a medical clinic or hospital.

Implantable analyte sensors allow for chemical analyte concentrations to be measured in real time even when a patient is away from a medical clinic or hospital and without the need to draw any fluids from the patient. This allows for a patient's chemical analyte concentrations to be monitored more frequently, for example continuously or semi-continuously, and allow physicians to gather larger data sets to be evaluated.

However, implantable analyte sensors require a positive immune system response to be effective. Embodiments herein, can include an implantable medical device coated, at least partly, in a fibrous cover layer. The fibrous cover layer can have high biochemical permeability and the texture and porosity can be tuned for optimal biocompatibility. The fibrous cover layer can also be used beneficially prevent direct interaction between the immune system and that which is covered by the fibrous cover layer such as an optical chemical sensor.

In some embodiments, the fibrous cover layer can be fabricated using an electrospinning process. Electrospinning allows the fibrous cover layer to achieve rapid diffusion and high permeability. Further, electrospinning allows for the fibrous cover layer to be tunable such that the fibrous cover layer can be modulated for analytes of interest. Tuning can include modulating the fiber diameter, the fiber deposition density, the fiber material composition, and the like.

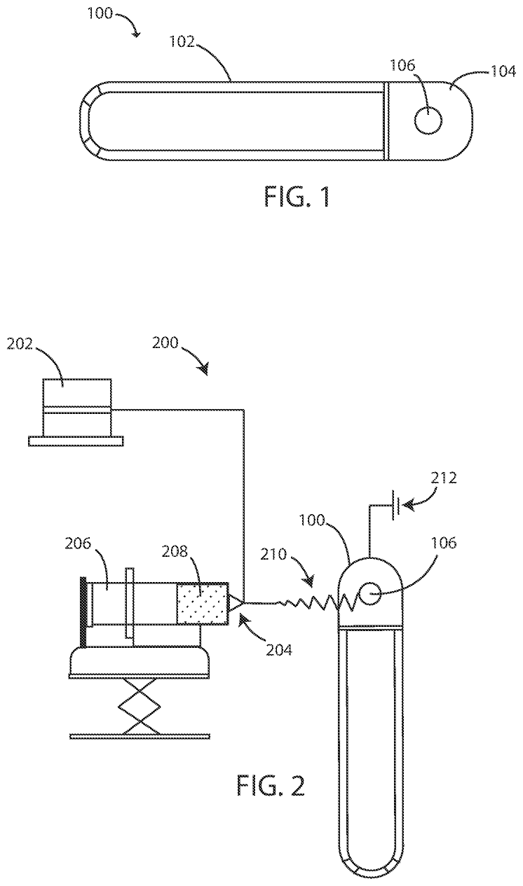

Referring now to FIG. 1 an implantable medical device 100 is shown in accordance with various embodiments herein. The implantable medical device 100 can include an implantable housing 102 and a header 104 coupled to the implantable housing 102. Various materials can be used. However, in some embodiments, the implantable housing 102 can be formed of a material such as a metal, ceramic, a polymer, or a composite. The header 104 can be formed of various materials, but in some embodiments the header 104 can be formed of a polymer (translucent or opaque) such as an epoxy material. In some embodiments the header 104 can be hollow. In other embodiments the header 104 can be filled with components and/or structural materials such as epoxy or another material such that it is non-hollow. In some embodiments, however, a distinct header 104 can be omitted. Rather, the implantable housing 102 can include substantially all of the components of the device.

The implantable medical device 100 can also include an optical chemical sensor 106. The optical chemical sensor 106 can be configured to detect an ion concentration of a bodily fluid when implanted in the body. Bodily fluids can include blood, interstitial fluid, serum, lymph, serous fluid, cerebrospinal fluid, and the like. In some embodiments the optical chemical sensor can be configured to detect one or more of an electrolyte, a protein, a sugar, a hormone, a peptide, an amino acid and a metabolic product. In some embodiments, the optical chemical sensor can be configured to detect an ion selected from a group consisting of potassium, sodium, chloride, calcium, magnesium, lithium, hydronium, hydrogen phosphate, bicarbonate, and the like. However, many other chemical analytes are also contemplated herein.

It will be appreciated that the optical chemical sensor 106 can be positioned at any location along implantable medical device 100, including along the implantable housing 102 or along the header 104. Additionally, it is noted the top of the optical chemical sensor 106 can be flush with the surface of the implantable housing 102 or alternatively, the top of the optical chemical sensor 106 can protrude from the surface of the implantable housing 102. It will also be appreciated that though FIG. 1 shows a device having one optical chemical sensor 106, any number of sensors can be present. For example, the device can include at least 1, 2, 3, 4, 5, 6, 7, 8, 9, 10, 11, 12, or more chemical sensors, or a number of chemical sensors falling within a range between any of the foregoing.

The implantable medical device 100 can take on various dimensions. In some embodiments herein it can be approximately 2 to 3 inches in length, 0.4 to 0.6 inches wide, and 0.15 to 0.35 inches thick. However, in some embodiments, the implantable medical device 100 can be about 0.25, 0.50, 1.0, 2.0, 3.0, 4.0, or 5.0 inches in length. In some embodiments the length can be in a range wherein any of the foregoing lengths can serve as the upper or lower bound of the range, provided that the upper bound is greater than the lower bound. In some embodiments, the implantable medical device 100 can be about 0.25, 0.50, 0.75, 1.0, or 2.0 inches in width. In some embodiments the length can be in a range wherein any of the foregoing lengths can serve as the upper or lower bound of the range, provided that the upper bound is greater than the lower bound. In some embodiments, the implantable medical device 100 can be about 0.10, 0.25, 0.50, 0.75, or 1.0 inches thick. In some embodiments the thickness can be in range wherein any of the foregoing thickness can serve as the upper or lower bound of the range, provided that the upper bound is greater than the lower bound.

In some embodiments, an electrospinning process can be used, in part, to create a fibrous cover layer. The fibrous cover layer can cover the implantable medical device 100 or one or more portions thereof. For example, in some embodiments the fibrous cover layer can cover the optical chemical sensor 106. Referring now to FIG. 2, an electrospinning process 200 is shown in accordance with various embodiments herein. The electrospinning process 200 can be used to create a fibrous cover layer coating the implantable medical device. FIG. 2 shows a power supply 202 that provides the power to produce an electric field between a polymer composition 208 at a tip 204 of a syringe 206 and a deposition substrate.

The deposition substrate in this example can include the implantable medical device 100 that is grounded 212 and/or limited portions thereof. The electric field created between the tip 204 and the implantable medical device 100 creates an electrostatic force that causes a surface tension of the droplet of the polymer composition 208 to be overcome. When the surface tension of the droplet of the polymer composition 208 is overcome by the electrostatic forces created, the droplet of the polymer composition 208 becomes a charged, continuous jet of electrospun fibers 210 that rapidly dry and thin in the air as the electrospun fibers 210 move toward the implantable medical device 100. The electrospun fibers 210 are deposited on the implantable medical device 100 as deposited fibers build up to form a layer. In some embodiments, the deposited fibers are arranged in a nonwoven, random orientation.

The electrospun fibers 210 can have a diameter of various dimensions. In some embodiments, the electrospun fibers can have a diameter of 500 nanometers to 1 micron. In some embodiments, the diameter can be greater than or equal to 100 nm, 275 nm, 450 nm, 625 nm, or 800 nm. However, in some embodiments, the diameter can be less than or equal to 2000 nm, 1700 nm, 1400 nm, 1300 nm, or 800 nm. In other embodiments, the diameter can fall within a range of 100 nm to 2000 nm, or 275 nm to 1700 nm, or 450 nm to 1400 nm, or 625 nm to 1300 nm, or can be about 800 nm. In some embodiments the diameter can be in range wherein any of the foregoing diameter can serve as the upper or lower bound of the range, provided that the upper bound is greater than the lower bound.

The electrospun fibers 210 can be deposited at various densities. One way of describing the density includes reference to the percentage of a given volume that is taken up by the fibers themselves versus the empty space between fibers. Such a percentage can also be used to calculate a density in units of weight per unit volume by multiplying the percentage as a decimal by the weight per unit volume of the material composition used to form the fibers. In various embodiments, the density can be at least 0.1, 0.5, 0.75, 1, 1.5, 2, 5, 10, 15, 20, 30, 40, 50, 60, 70, 80 or 90 percent or higher, or a density falling within a range between any of the foregoing.

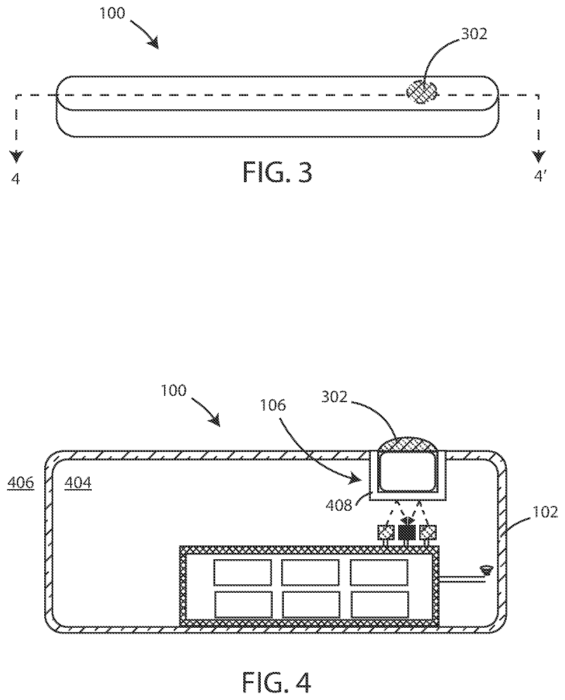

In some embodiments, a fibrous cover layer can be disposed over the chemical sensor of the implantable medical device. Referring now to FIG. 3, an implantable medical device 100 coated with a fibrous cover layer 302 is shown in accordance with various embodiments herein. It will be appreciated that having a fibrous cover layer 302 disposed over the optical chemical sensor of the implantable medical device 100 can provide various benefits such as protecting components of the optical chemical sensor while allowing for the interface in the area of the optical chemical sensor with the in vivo environment to be optimized.

Referring now to FIG. 4, a cross-sectional view (not to scale) is shown of an implantable medical device coated with a fibrous cover layer as taken along line 4-4′ of FIG. 3 in accordance with various embodiments herein. As shown, the implantable medical device can include a housing 102 defining an interior volume 404 and can also include a sensor optical carrier 408 attached to the housing 102. The implantable housing 102 can separate the interior volume 404 of the implantable medical device 100 from the surrounding in vivo environment 406 after the implantable medical device 100 has been implanted.

The sensor optical carrier 408 or portions thereof can be transparent or at least semi-transparent can be formed of a polymer or a glass. Some components of the optical chemical sensor (such as the sensor element(s) or tag(s) thereof) can be disposed within a well that is defined by the sensor optical carrier 408.

The fibrous cover layer 302 can be disposed over the optical chemical sensor 106. It will be appreciated that in this example the fibrous cover layer 302 protrudes outward from the optical chemical sensor 106, but exhibits rounded edges to allow for easy insertion of the implantable medical device 100 into a human body. However, other physical configurations of the fibrous cover layer 302 are also contemplated herein.

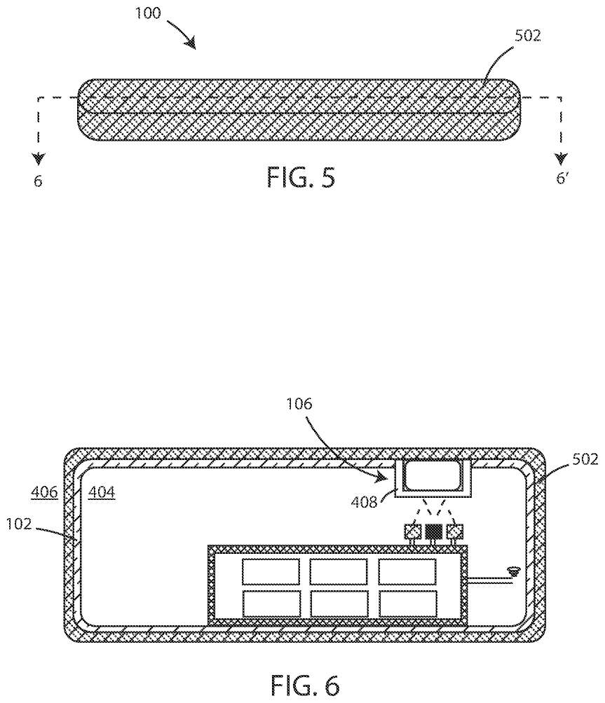

In some embodiments, portions of the implantable medical device can be covered in a fibrous layer other than just the area over the optical chemical sensor. Referring now to FIG. 5, an implantable medical device coated with a fibrous cover layer is shown in accordance with various embodiments herein. In some embodiments, a fibrous cover layer 502 can be deposited onto the implantable medical device 100 using the electrospinning process 200. Exemplary materials for the fibrous cover layer 502 are described in greater detail below.

In some embodiments, the fibrous cover layer 502 can be evenly disposed over the entire implantable medical device 100. In some embodiments, the fibrous cover layer 502 can encapsulate the entire implantable medical device 100, including encapsulating the implantable housing 102, the header 104, and the optical chemical sensor 106. However, in other embodiments, the fibrous cover layer 502 may only cover some portions of the implantable medical device 100. For example, the fibrous cover layer 502 can cover the implantable housing 102, in full or in part, the header 104, in full or in part, and/or the optical chemical sensor 106, in full or in part.

Referring now to FIG. 6, a cross-sectional view of an implantable medical device coated with a fibrous layer as taken along line 6-6′ of FIG. 5 is shown in accordance with various embodiments herein. As shown, the implantable medical device includes a housing 102 defining an interior volume 404 along with a sensor optical carrier 408 attached to the housing 102. The sensor optical carrier 408 can include some components of the optical chemical sensor 106. The implantable housing 102 can separate the interior volume 404 from the in vivo environment 406. As shown, the fibrous cover layer 502 can be evenly disposed over the implantable housing 102. In some embodiments, the fibrous cover layer 502 can encapsulate the implantable housing 102.

The fibrous cover layer 502 can be of varying thickness. In various embodiments, the thickness of the fibrous cover layer 502 should be sufficiently thin enough to allow for the rapid diffusion of various analytes into the implantable medical device 100. However, the fibrous cover layer 502 should be sufficiently thick so as to provide protection for the optical chemical sensor 106 and/or to provide a desirable interface with the in vivo environment.

In various embodiments, the fibrous cover layer 502 can take on various dimensions. In some embodiments herein the fibrous cover layer 502 can be from 10 microns to 2 millimeters thick. However, in some embodiments, the fibrous cover layer 502 can be about 10, 20, 30, 40, 50, 75, 100, 125, 150, 200, 250, 500, 750, 1000, 1250, 1500, 1750, 2000, 2250, 2500, 2750, 3000 microns thick. In some embodiments the thickness can be in a range wherein any of the forgoing thicknesses can serve as the upper or lower bound of the range, provided that the upper bound is greater than the lower bound.

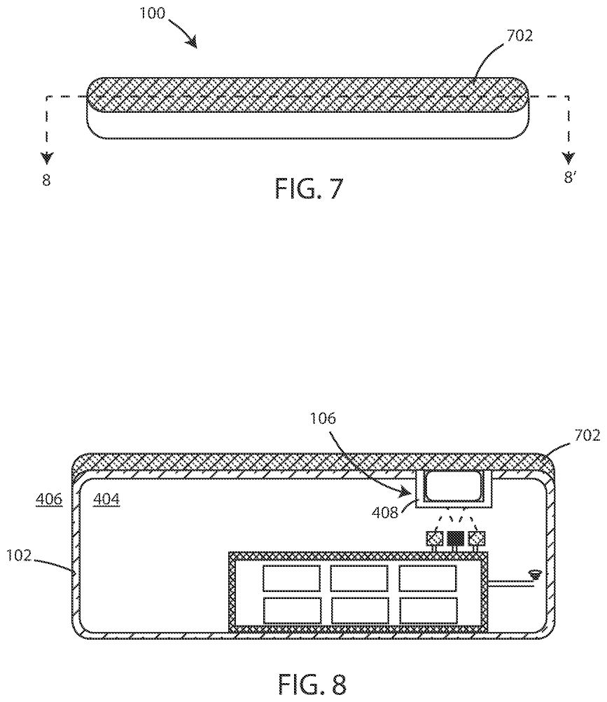

In some embodiments, the fibrous cover layer 502 can be disposed over one or more surfaces of the implantable medical device 100 as opposed to being disposed over the entire implantable medical device 100 or just the optical chemical sensor as discussed above. Referring now to FIG. 7, an implantable medical device 100 coated with a fibrous cover layer 702 is shown in accordance with various embodiments herein. In some embodiments, the fibrous cover layer 702 can be disposed over a particular surface of the implantable medical device 100. As shown, one side/surface of the implantable medical device 100 is coated in the fibrous cover layer 702.

Coating a particular surface/side can offer various potential advantages. For example, manufacturing can be eased by coating the entire surface/side on which the optical chemical sensor is disposed instead of just over the optical chemical sensor itself. Also, the fibrous cover layer 702 can be secured more tightly and prevented from peeling back upon encountering friction (such as that which may occur during the implantation process). Also, in some embodiments, by covering an entire surface/side of the implantable medical device 100, the different sides of the implantable medical device 100 can be made more apparent to the naked eye to make it easier to implant the implantable medical device with a particular side facing inward toward the center of the body of the patient versus outward toward the skin of the patient (such as in a subcutaneous implant scenario). In order to facilitate visual recognition of one side or another of the implantable medical device 100, the fibers of the fibrous cover layer 702 can be made a particular color such as red, green, blue or the like so that the fibrous cover layer 702 is visually distinct from exterior portions of the housing of the implantable medical device 100. In some embodiments, the fibrous cover layer 702 can be used to provide optical shielding. For example, the fibrous cover layer 702 can include pigments that can be used to minimize external optical interference from environmental light. Pigments can include various types of pigments including black pigments, infrared blocking pigments, and the like.

Referring now to FIG. 8, a cross-sectional view of an implantable medical device 100 coated with a fibrous cover layer 702 taken along line 8-8′ of FIG. 7 is shown in accordance with various embodiments. The implantable medical device 100 can include implantable housing 102. The implantable housing 102 can define interior volume 404 and the sensor optical carrier 408 can be attached to the implantable housing 102. The sensor optical carrier 408 can hold some components of the optical chemical sensor 106. The fibrous cover layer 702 can be disposed over a surface/side of the implantable medical device including the surface/side having the sensor optical carrier 408 and the optical chemical sensor 106.

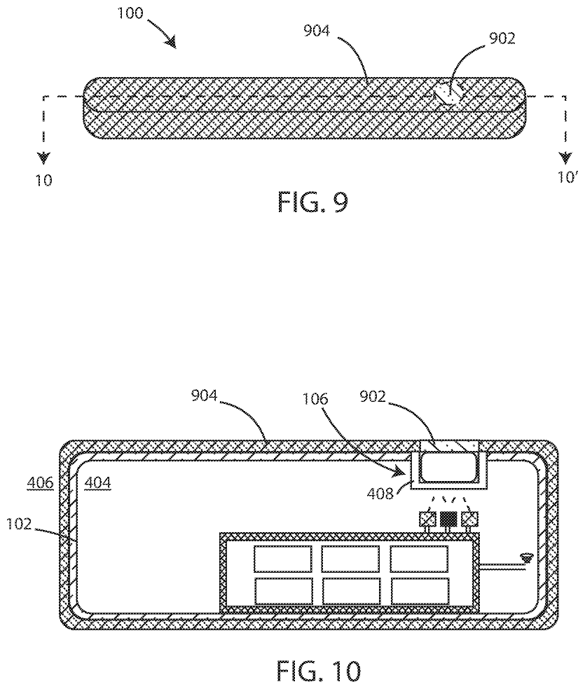

In some embodiments, the fibrous cover layer can be disposed over one or more portions of the implantable medical device in varying thicknesses, densities, fiber diameters, and/or materials. For example, the fibrous cover layer can include at least two different portions that are different from one another in various ways and by different degrees such as at least 10, 20, 30, 40, 50, 75, 100, 200 percent different or more. In this way, different portions can be tuned to be optimized for the potentially different functions of different parts of the implantable medical device. Referring now to FIG. 9, an implantable medical device coated with fibrous layers is shown in accordance with various embodiments herein. The implantable medical device 100 can include a first fibrous cover layer zone 902 (or first fibrous cover layer portion) disposed over a portion of the implantable medical device 100. Further, a second fibrous cover layer zone 904 (or second fibrous cover layer portion) can be disposed over a second portion of the implantable medical device 100. For example, the first fibrous cover layer zone 902 can be disposed over the optical chemical sensor of the implantable medical device 100 and the second fibrous cover layer zone 904 can be disposed over the implantable housing (not shown in this view). Many other configurations are also contemplated herein.

In various embodiments, the zones 902, 904 can have a different thickness, fiber density, fiber size, or fiber composition from one another. It can be appreciated that varying the thickness, fiber density, fiber size, fiber composition, electrical conductivity, and/or permittivity of the zones can be desirable. For example, in some embodiments, it can be desirable to modulate the fiber diameter, density and/or material in the zone of the optical chemical sensor to facilitate tissue in-growth that may promote rapid diffusion of analytes into the optical chemical sensor whereas it can be desirable to modulate the fiber diameter, density, and/or material in the area of the increase the fiber thickness in the zone of the rest of the implantable housing to prevent tissue in-growth that may make later removal of the implantable medical device 100 more difficult. In some embodiments, the implantable medical device can include one or more electrodes on the surface thereof and the one or more zones can be oriented such that a zone exhibiting greater conductivity can be aligned with the one or more electrodes. In some embodiments, zones can be oriented such that the ends of the implantable device are covered by one zone while the rest of the implantable device is covered by another zone. The different zones can be formed in various ways. In some embodiments, they can be formed by adjusting parameters and/or materials used in an electrospinning or electrospraying process. In some embodiments, they can be formed using a selective masking approach.

Referring now to FIG. 10, a cross-sectional view of an implantable medical device coated with a fibrous layer taken along line 10-10′ of FIG. 9 is shown in accordance with various embodiments herein. The implantable medical device includes an interior volume 404 and sensor optical carrier 408. Some components of the optical chemical sensor 106 can be disposed within the sensor optical carrier 408. The implantable housing 102 can separate the interior volume 404 from the in vivo environment 406. The first fibrous cover layer zone 902 and the second fibrous cover layer zone 904 can cover different portions of the implantable medical device. As discussed above, the first fibrous cover layer zone 902 and the second fibrous cover layer zone 904 can have a different thickness, fiber density, fiber size, or fiber composition from one another.

In some embodiments, instead of depositing fibers directly onto the implantable medical device, fibers can be deposited onto a substrate (such as a frame or other device). Then, after deposition onto the substrate, the fibrous layer can be removed from the substrate and transferred onto the implantable medical device or, in the case of a structure such as a frame, the frame itself can be disposed over or placed onto the housing of the medical device such that the frame and the fibrous layer it bears become part of the implantable medical device.

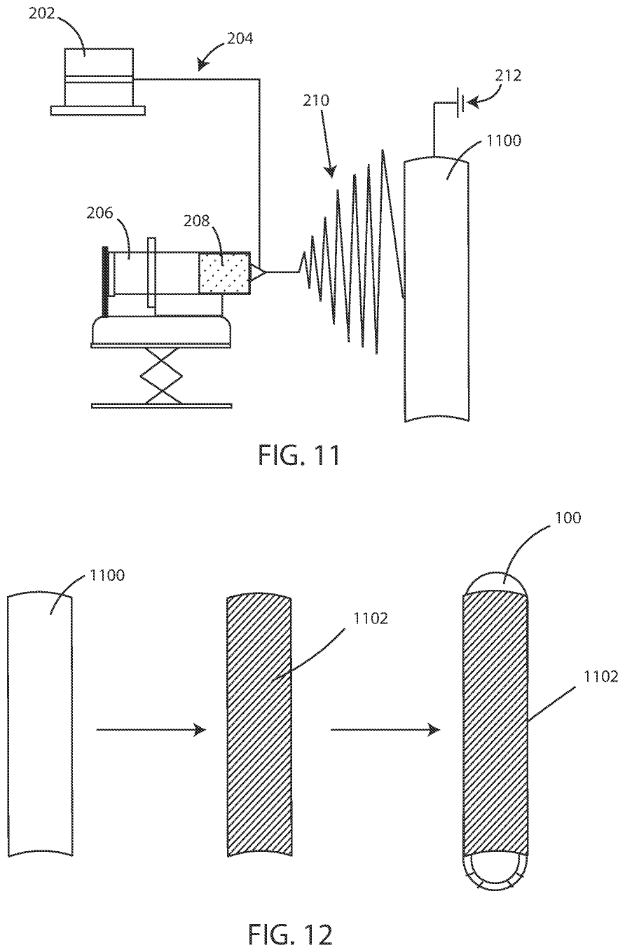

Referring now to FIG. 11, a fibrous cover layer being spun onto a frame is shown in accordance with various embodiments herein. Using an electrospinning process, electrospun fibers 210 can be deposited onto the frame 1100. Later, such as shown in FIG. 12, the frame 1100 can be placed on/over the implantable medical device 100.

It can be appreciated that electrospinning fibers onto the frame 1100 can protect the implantable medical device 100 from damage that could possibly otherwise occur during the electrospinning process. Further, electrospinning deposited fibers onto the frame 1100 can facilitate efficient manufacturing by allowing for large quantities of frames 1100 coated in deposited fibers to be fabricated separate from the implantable medical devices 100 themselves. In addition, electrospinning deposited fibers onto the frame 1100 can allow for more rapid, modular production of devices that are tuned for different sensing scenarios and/or device placement. For example, a selection can be made of a particular frame 1100 from amongst a group of frames to select for deposited fiber layer characteristics (fiber type, fiber diameter, fiber density, layer thickness, etc.) that are ideal for a given scenario or planned device placement and then that particular frame 1100 can be placed on/over an implantable medical device 100 to customize it for the specific scenario/placement.

In some embodiments, the fibers can be deposited onto a form that is similar in shape to the implantable medical device. Then, the fibrous layer can be rolled down the form and then transferred to and rolled up the implantable medical device.

FIG. 12 shows stages of a process wherein fibers are deposited onto a frame 1100 first and then the frame is fit over/fit on the implantable medical device. FIG. 12 also shows a frame 1100 coated with a fibrous cover layer 1102 being placed onto an implantable medical device after the fibrous cover layer 1102 was deposited onto the frame 1100. In some embodiments, the frame 1100 can be the same shape as the implantable medical device 100. In other embodiments, the frame 1100 can come in various shapes that still allow for the frame 1100 to be placed over the implantable medical device 100. For example, the frame 1100 can be rectangular, conical, columnar, pyramidal, polygonal, or the like. The frame 1100 can cover the whole implantable medical device or only portions thereof.

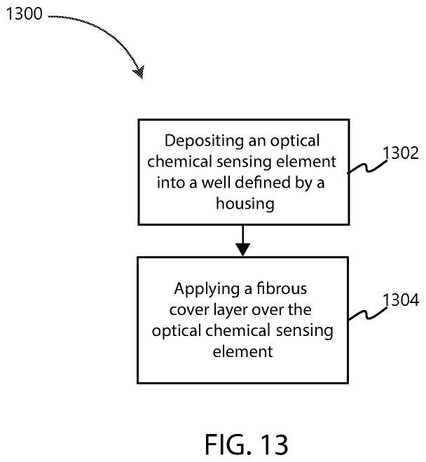

Referring now to FIG. 13, a flow diagram of a method of making an implantable medical device is shown in accordance with various embodiments herein. The method 1300 can include an operation of a depositing 1302 an optical chemical sensing element (or tag) into a sensor optical carrier, which can be attached to the housing of the implantable medical device. In some embodiments, the sensor optical carrier can include a plurality of optical chemical sensing elements.

The method 1300 can include an operation of applying 1304 a fibrous cover layer over the chemical sensing element. In some embodiments, the fibrous cover layer can be deposited using an electrospinning process. However, in other embodiments, the fibrous cover layer may not be fibrous, but rather a polymeric cover layer that can be deposited using a different deposition process including a conventional spraying process (including, but not limited to electrospraying), dip coating, blade coating, print deposition, or the like. In some embodiments, the fibrous cover layer can be formed from a hydrophobic polymer. In some embodiments, the fibrous cover layer can be formed from a hydrophilic polymer. However, other polymers are also contemplated as described further below. The fibrous cover layer can be porous and/or allow for tunable, rapid diffusion of analytes into the implantable medical device. In will be appreciated that various other operations can be performed in between operation 1302 and operation 1304.

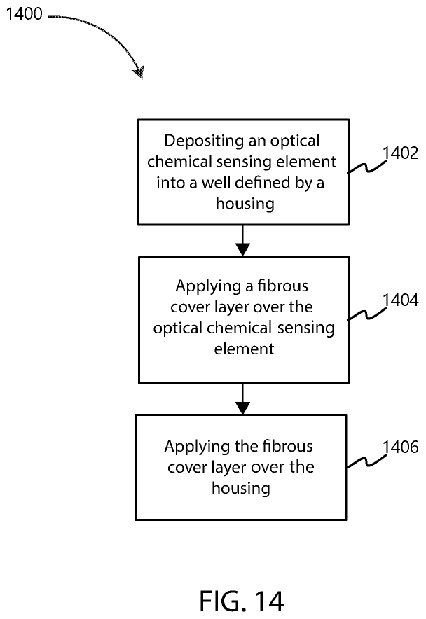

Referring now to FIG. 14, a flow diagram of a method of making an implantable medical device is shown in accordance with various embodiments herein. As before, the method 1400 can include operations of depositing 1402 an optical chemical sensing element into a sensor optical carrier and applying 1404 a fibrous cover layer over the optical chemical sensing element. The method 1400 can further include applying 1406 the fibrous cover layer over the housing of an implantable medical device.

In some embodiments, the fibrous cover layer can encapsulate the housing. In some embodiments, the fibrous cover layer applied over the optical chemical sensing element can have a different thickness, fiber density, fiber size, or fiber composition from the fibrous cover layer applied over the housing. In other embodiments, the fibrous cover layer applied over the housing and the fibrous cover layer applied over the optical chemical sensing element can have the same thickness, fiber density, fiber size, or fiber composition.

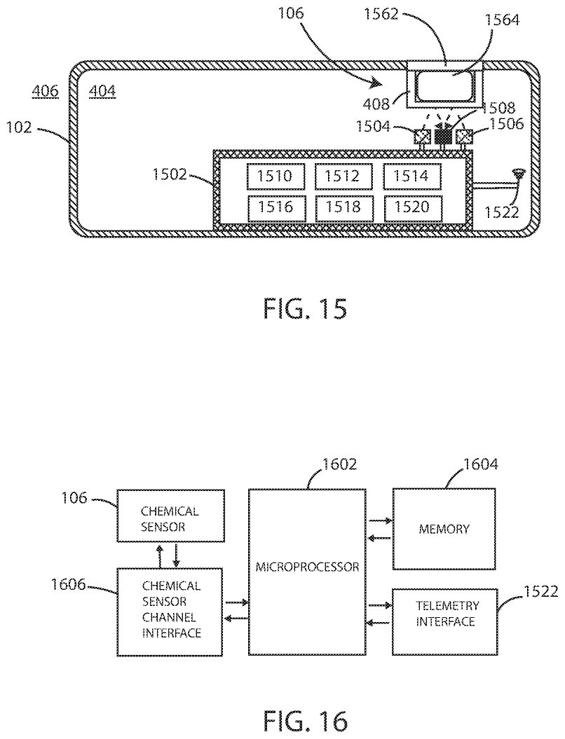

It will be appreciated that implantable medical devices herein can include many other components beyond the fibrous cover layer. Referring now to FIG. 15, a schematic view of implantable medical device 100 is shown in accordance with various embodiments herein. FIG. 15 shows the device without a fibrous cover layer, though it will be appreciated that this is just for ease of explanation and that the device can include a fibrous cover layer as described elsewhere herein. The implantable medical device 100 can include implantable housing 102. The implantable housing 102 of the implantable medical device 100 can include various materials such as metals, polymers, ceramics, and the like. In some embodiments, the implantable housing 102 can be a single integrated unit. In other embodiments, the implantable housing 102 can include implantable housing 102 and header 104, as discussed above. In some embodiments, the implantable housing 102, or one or more portions thereof, can be formed of titanium.

The implantable housing 102 can define an interior volume 404 that in some embodiments is hermetically sealed off from the in vivo environment 406 outside of the implantable medical device 100. The implantable medical device 100 can include sensor optical carrier 408 which can hold one or more optical chemical sensor elements 1564, as discussed above. In some embodiments, the sensor optical carrier 408 can be covered by a non-fibrous cover layer 1562 (separate from the fibrous cover layer). In other embodiments, the non-fibrous cover layer 1562 can be omitted.

The chemical sensor elements or tags can include a chemical sensing composition used to provide an optical response based on the presence of one or more chemical analytes. Exemplary chemical sensing compositions are described in greater detail below.

The non-fibrous cover layer 1562 can be formed from a permeable material, such as an ion permeable polymeric matrix material. In some embodiments, the non-fibrous cover layer 1562 can be permeable to one or more of potassium, sodium, chloride, calcium, magnesium, lithium, hydronium, hydrogen phosphate, bicarbonate. Many different materials can be used as the ion permeable polymeric matrix material. In some embodiments, the ion permeable polymeric matrix material can be a hydrogel. In some embodiments, the ion permeable polymeric material can be polyhydroxyethyl methacrylate (polyHEMA) either as a homopolymer or a copolymer including the same. The ion permeable polymeric matrix material(s) can be chosen based on its permeability to one or more of an electrolyte, a protein, a sugar, a hormone, a peptide, an amino acid, or a metabolic product. Specific ion permeable polymeric matrix material are discussed in more detail below. In some embodiments, the non-fibrous cover layer 1562 can be opaque to the passage of light in one or more of the visible, ultraviolet (UV), or near-infrared (NIR) frequency spectrums.

The implantable medical device 100 can further include circuitry 1502. The circuitry 1502 can include various components, such as components 1510, 1512, 1514, 1516, 1518, 1520. In some embodiments, these components can be integrated and in other embodiments these components can be separate. In some embodiments, the components can include one or more of control circuitry (microprocessor, microcontroller, an ASIC, or the like), memory circuitry (such as random access memory (RAM) and/or read only memory (ROM)), recorder circuitry, telemetry circuitry, chemical sensor interface circuitry, power supply circuitry (which can include one or more batteries), normalization circuitry, chemical sensor control circuitry, and the like. In some embodiments, recorder circuitry can record the data produced by the chemical sensor and record time stamps regarding the same. In some embodiments, the circuitry can be hardwired to execute various functions, while in other embodiments the circuitry can be implemented as instructions executing on a microprocessor or other computation device.

A telemetry interface 1522 can be provided for communicating with external devices such as a programmer, a home-based unit, and/or a mobile unit (e.g., a cellular phone, portable computer, etc.). In some embodiments, the telemetry interface 1522 can be provided for communicating with implanted devices such as a therapy delivery device (e.g. a pacemaker, cardiovertor-defibrillator) or monitoring only device (e.g. an implantable loop recorder). In some embodiments, the circuitry can be implemented remotely, via either near-field, far-field, conducted, intra-body or extracorporeal communication, from instructions executing on any of the external or the implanted devices, etc. In some embodiments, the telemetry interface 1522 can be located within the implantable housing 102. In some embodiments, the telemetry interface 1522 can be located in header 104.

An optical excitation assembly 1508 as well as optical detection assemblies 1504, 1506 can be in electrical communication with the circuitry 1502 within the interior volume 404. In some embodiments, the circuitry 1502 is configured to selectively activate the optical excitation assembly 1508 and optical detection assemblies 1504, 1506. The optical excitation assembly 1508 can be configured to cast light onto one or more chemical sensing elements disposed within the sensor optical carrier 408. The optical detection assemblies 1504, 1506 are configured to receive light from the one or more chemical sensor elements.

While FIG. 15 shows light rays reflecting and/or absorbing near the bottom of the sensor optical carrier/sensing elements, it will be appreciated that this is merely for ease of illustration and that the entire volume of the sensor optical carrier and sensing element(s) therein can be exposed to light from the optical excitation assemblies and therefore contribute to generating a response that can be detected by the optical detection assemblies.

Referring now to FIG. 16, a schematic diagram of components of implantable medical device 100 in accordance with various embodiments herein. It will be appreciated that some embodiments can include additional elements beyond those shown in FIG. 16. In addition, some embodiments may lack some elements shown in FIG. 16. The implantable medical device 100 can gather information through one or more sensing channels. A microprocessor 1602 can communicate with a memory 1604 via a bidirectional data bus. The memory 1604 can include read only memory (ROM) or random access memory (RAM) for program storage and RAM for data storage, or any combination thereof. The implantable medical device 100 can also include one or more optical chemical sensors 106 and one or more chemical sensor channel interfaces 1606 which can communicate with a port of the microprocessor 1602. The chemical sensor channel interface 1606 can include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like. A telemetry interface 1522 is also provided for communicating with external devices such as a programmer, a home-based unit, and/or a mobile unit (e.g., a cellular phone, portable computer, etc.), implanted devices such as a pace maker, cardiovertor-defibrillator, loop recorder, and the like.

### Methods

Many different methods are contemplated herein, including, but not limited to, methods of making, methods of using, and the like. Aspects of system/device operation described elsewhere herein can be performed as operations of one or more methods in accordance with various embodiments herein.

In an embodiment, a method of making an implantable medical device is included, the method applying a fibrous cover layer over a frame member and fitting the frame member over at least one of a housing and a header of the implantable medical device.

In an embodiment of the method, applying a fibrous cover layer over a frame member further comprises electrospinning the fibrous cover layer over the frame member. In other embodiments of the method, the fibrous cover layer can be sprayed, dip coated, blade coated, painted/brushed onto the frame, or any other appropriate method to apply onto the frame. In other embodiments, the fibrous cover layer can be sprayed, dip coated, painted/brushed onto the implantable medical device.

In an embodiment of the method, fitting the frame member over at least one of a housing and a header of the implantable medical device further comprises the frame member having the approximate shape of the implantable medical device. In some embodiments, the frame can be cylindrical in shape. In other embodiments, the frame can be rectangular, conical, columnar, pyramidal, polygonal, or the like.

In an embodiment, the method can further include applying the fibrous cover layer over the optical chemical sensing element. In some embodiments, the fibrous cover layer can be deposited over the optical chemical sensing element and the implantable housing. In other embodiments, the fibrous cover layer can be deposited over the optical chemical sensing element. Alternatively, the fibrous cover layer can be deposited over the implantable housing.

In some embodiments, the fibrous cover layer deposited over the optical chemical sensing element and the implantable housing can have the same thickness, fiber density, fiber size, or fiber composition. In other embodiments, the fibrous cover layer over the optical chemical sensing element can have a different thickness, fiber density, fiber size, or fiber composition from the fibrous cover layer over the implantable housing.

In an embodiment, the method can further include the fibrous cover layer encapsulating the implantable housing. In other embodiments, the fibrous cover layer can cover a portion of the implantable housing.

### Fibrous Cover Layer

Various embodiments herein include a fibrous cover layer. Further details about the fibrous cover layer are provided as follows. However, it will be appreciated that this is merely provided by way of example and that further variations are contemplated herein.

As referenced above, the fibrous cover layer of the implantable medical device can be formed of a biocompatible thermoplastic polymer. Suitable polymers for use as biocompatible, biostable thermoplastic polymers can include, but are not limited to homopolymeric thermoplastics, copolymeric thermoplastics, polymeric alloys multipolymer interpenetrating polymeric thermoplastics, and any polymers capable of being electrospun.

In some embodiments, biocompatible thermoplastics herein can include, but are limited to polyvinylchloride (PVC), polysulfone (PS), polytetrafluorethylene (PTFE), polyethylene (PE), polypropylene (PP), polyethersulfone (PES), polyurethane (PU), polyetherimide (PEI), polycarbonate (PC), polyetheretherketone (PEEK), cellulose, polyethylene glycol (PEG), and the like.

In some embodiments the biocompatible thermoplastic can include biocompatible thermoplastic polymers infused with plasticizers to increase the flexibility and plasticity of the biocompatible thermoplastic while decreasing its viscosity and friction. Plasticizers can include one or more of bis(2-ethylhexyl) phthalate (DEHP), diisooctyl phthalate (DIOP), acetyl tributyl citrate (ATBC), acetyl trihexyl citrate (ATHC), butyryl trihexyl citrate (BTHC), trimethyl citrate (TMC), and the like.

In some embodiments, the biocompatible thermoplastic polymers described above can include polyethylene glycol molecules covalently bonded to the biocompatible thermoplastic polymers. In other embodiments, polyethylene glycol subunits can be copolymerized with the biocompatible thermoplastic polymers described above.

In some embodiments, the fibrous cover layer can exist in the form of fibers, such as would be the result of an electrospinning process. In some embodiments, the fibers can specifically include polyvinyl chloride and a plasticizer. The fibers can be of various diameters. In some embodiments, the fibers can have an average diameter of greater than or equal to 100 nm, 200 nm, 300 nm, 400 nm, 500 nm, or 600 nm. In some embodiments, the average diameter can be less than or equal to 2000 nm, 1720 nm, 1440 nm, 1160 nm, 880 nm, or 600 nm. In some embodiments, the average diameter can fall within a range of 100 nm to 2000 nm, or 200 nm to 1720 nm, or 300 nm to 1440 nm, or 400 nm to 1160 nm, or 500 nm to 880 nm, or can be about 600 nm.

### Chemical Sensing Composition

Various embodiments herein include a chemical sensing composition that can be within a chemical sensor element or tag. Further details about the chemical sensing composition are provided as follows. However, it will be appreciated that this is merely provided by way of example and that further variations are contemplated herein.

As referenced above, the chemical sensing composition of the chemical sensor element can be formed of a lipophilic indicator dye. In some embodiments, the chemical sensing composition can be formed from a lipophilic fluorescent indicator dye. In other embodiments, the chemical composition can be formed from a lipophilic colorimetric indicator dye. Suitable lipophilic indicator dyes can include, but are not limited to, ion selective sensors such as ionophores or fluorophores.

In some embodiments, ionophores herein can include, but not be limited to, sodium specific ionophores, potassium specific ionophores, calcium specific ionophores, magnesium specific ionophores, and lithium specific ionophores. In some embodiments, fluorophores can include, but not be limited to, lithium specific fluorophores, sodium specific fluorophores, and potassium specific fluorophores.

Chemical sensing compositions herein can include components (or response elements) that are configured for a colorimetric response, a photoluminescent response, or another optical sensing modality.

Colorimetric response elements herein can be specific for a particular chemical analyte. Colorimetric response elements can include an element that changes color based on binding with or otherwise complexing with a specific chemical analyte. In some embodiments, a colorimetric response element can include a complexing moiety and a colorimetric moiety. Those moieties can be a part of a single chemical compound (as an example a non-carrier based system) or they can be separated on two or more different chemical compounds (as an example a carrier based system). The colorimetric moiety can exhibit differential light absorbance on binding of the complexing moiety to an analyte.

Photoluminescent response elements herein can be specific for a particular chemical analyte. Photoluminescent response elements herein can include an element that absorbs and emits light through a photoluminescent process, wherein the intensity and/or wavelength of the emission is impacted based on binding with or otherwise complexing with a specific chemical analyte. In some embodiments, a photoluminescent response element can include a complexing moiety and a fluorescing moiety. Those moieties can be a part of a single chemical compound (as an example a non-carrier based system) or they can be separated on two or more different chemical compounds (as an example a carrier based system). In some embodiments, the fluorescing moiety can exhibit different fluorescent intensity and/or emission wavelength based upon binding of the complexing moiety to an analyte.

Some chemistries may not require a separate compound to both complex an analyte of interest and produce an optical response. By way of example, in some embodiments, the response element can include a non-carrier optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy)thiophenyl]coumarin, a potassium ion selective fluoroionophore, can be used (and in some cases covalently attached to polymeric matrix or membrane) to produce a fluorescence-based K⁺ non-carrier response element.

An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″-carboethoxy)furyl]coumarin. Sodium specific fluoroionophores can include (6,7-[2.2.1]-cryptando-3-[2″-(5″-carboethoxy)furyl]coumarin. Potassium specific fluoroionophores can include (6,7-[2.2.2]-cryptando-3-[2″-(5″-carboethoxy)furyl]coumarin and (6,7-[2.2.2]-cryptando-3-[2″-(5″-carboethoxy)thiophenyl]coumarin.

Suitable fluoroionophores include the coumarocryptands taught in U.S. Pat. No. 5,958,782, the disclosure of which is herein incorporated by reference. Such fluorescent ionophoric compounds can be excited with GaN blue light emitting diodes (LEDs) emitting light at or about 400 nm. These fluorescent ionophoric compounds have ion concentration dependent emission that can be detected in the wavelength range of about 450 nm to about 470 nm.

Some chemistries can rely upon a separate complexing entity (e.g., a separate chemical compound). As an example, carrier based response elements can include a compound, in some cases referred to as an ionophore, that complexes with and serves to carry the analyte of interest. In some embodiments, carrier based response elements include a lipophilic ionophore, and a lipophilic fluorescent or colorimetric indicator dye, called a chromoionophore. In some cases the chromoionophore and the ionophore can be dispersed in, and/or covalently attached to, a hydrophobic organic polymeric matrix. The ionophore can be capable of reversibly binding ions of interest. The chromoionophore can be a proton selective dye. In operation, analytes of interest are reversibly sequestered by the ionophores within the organic polymer matrix. To maintain charge neutrality within the polymer matrix, protons are then released from the chromoionophore, giving rise to a color or fluorescence change. As just one specific example, a carrier based response element can include potassium ionophore III, chromoionophore I, and potassium tetrakis(4-chlorophenyl)borate dispersed in a polymer matrix made from polyvinylchloride and bis(2-ethylhexyl)sebacate surfactant to produce a colorimetric K⁺ sensing element.

Both non-carrier based response elements and carrier-based response elements can include complexing moieties. Suitable complexing moieties can include cryptands, crown ethers, bis-crown ethers, calixarenes, noncyclic amides, and hemispherand moieties as well as ion selective antibiotics such as monensin, valinomycin and nigericin derivatives.

Those of skill in the art can recognize which cryptand and crown ether moieties are useful in complexing particular cations, although reference can be made to, for example, Lehn and Sauvage, “[2]-Cryptates: Stability and Selectivity of Alkali and Alkaline-Earth Macrocyclic Complexes,” J. Am. Chem. Soc, 97, 6700-07 (1975), for further information on this topic. Those skilled in the art can recognize which bis-crown ether, calixarene, noncyclic amides, hemispherand, and antibiotic moieties are useful in complexing particular cations, although reference can be made to, for example, Buhlmann et al., “Carrier-Based Ion-Selective Electrodes and Bulk Optodes. 2. Ionophores for Potentiometric and Optical Sensors,” Chem. Rev. 98, 1593-1687 (1998), for further information on this topic.

By way of example cryptands can include a structure referred to as a cryptand cage. For cryptand cages, the size of the cage is defined by the oxygen and nitrogen atoms and the size makes cryptand cages quite selective for cations with a similar diameter. For example a [2.2.2] cryptand cage is quite selective for cations such as K⁺, Pb⁺², Sr⁺², and Ba⁺². A [2.2.1] cryptand cage is quite selective for cations such as Na+ and Ca⁺². Finally, a [2.1.1] cryptand cage is quite selective for cations such as Li⁺ and Mg⁺². The size selectivity of cryptand cages can aid in the sensitivity of chemical sensing. When these cryptand cages are incorporated into physiologic sensing systems heavier metals such as Pb⁺² and Ba⁺² are unlikely to be present in concentrations which interfere with the analysis of ions of broader physiological interest such as Na⁺ and K⁺.

Further aspects of chemical sensor compositions are described in U.S. Pat. Nos. 7,809,441 and 8,126,554, the content of which is herein incorporated by reference.

### Optical Excitation and Detection Assemblies

In some embodiments, optical excitation assemblies herein can include solid state light sources such as GaAs, GaAlAs, GaAlAsP, GaAIP, GaAsp, Gap, GaN, InGaAIP, InGaN, ZnSe, or SiC light emitting diodes or laser diodes that excite the chemical sensor element(s) at or near the wavelength of maximum absorption for a time sufficient to emit a return signal. However, it will be understood that in some embodiments the wavelength of maximum absorption reflection varies as a function of concentration in the colorimetric sensor.

In some embodiments, the optical excitation assemblies can include other light emitting components including incandescent components. In some embodiments, the optical excitation assemblies can include a wave guide. The optical excitation assembly can also include one or more bandpass filters, high pass filter, low pass filter, antireflection elements, and/or focusing optics.

In some embodiments, the optical excitation assembly can include a plurality of LEDs with bandpass filters, each of the LED-filter combinations emitting at a different center frequency. According to various embodiments, the LEDs can operate at different center-frequencies, sequentially turning on and off during a measurement, illuminating the chemical sensor element. As multiple different center-frequency measurements are made sequentially, a single unfiltered detector can be used in some embodiments. However, in some embodiments, a polychromatic source can be used with multiple detectors that are each bandpass filtered to a particular center frequency.

The optical detection assemblies can be configured to receive light from the chemical sensor element. In an embodiment, the optical detection assemblies can include a component to receive light. By way of example, in some embodiments, the optical detection assemblies can include a charge-coupled device (CCD). In other embodiments, the optical detection assemblies can include a photodiode, a junction field effect transistor (JFET) type optical sensor, or a complementary metal-oxide semiconductor (CMOS) type optical sensor. In some embodiments, the optical detection assemblies can include an array of optical sensing components. In some embodiments, the optical detection assemblies can include a waveguide.

The optical detection assemblies can also include one or more bandpass filters and/or focusing optics. In some embodiments, the optical detection assemblies can include one or more photodiode detectors, each with an optical bandpass filter tuned to a specific wavelength range.

The optical excitation and detection assemblies respectively, can be integrated using bifurcated fiber-optics that direct excitation light from a light source to one or more chemical sensor elements, or simultaneously to chemical sensor element(s) and a reference channel. Return fibers can direct emission signals from the chemical sensor element(s) and the reference channels to one or more optical detector assemblies for analysis by a processor, such as a microprocessor. In some embodiments, the optical excitation and detection assemblies are integrated using a beam splitter assembly and focusing optical lenses that direct excitation light from a light source to the sensing element and direct emitted or reflected light from the sensing element to an optical detector for analysis by a processor.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it it should be understood that many variations and modifications may be made while remaining within the spirit and scope herein.

1. An implantable medical device comprising:
a housing;
an optical chemical sensing element, wherein the optical chemical sensing element is disposed along the housing; and
a fibrous electrospun cover layer, wherein the fibrous electrospun cover layer is disposed over the optical chemical sensing element.

2. The implantable medical device of claim 1, wherein the fibrous electrospun cover layer is from 10 microns to 2 millimeters thick.

3. The implantable medical device of claim 1, wherein the fibrous electrospun cover layer is disposed over the optical chemical sensing element and the housing.

4. The implantable medical device of claim 1, wherein the fibrous electrospun cover layer encapsulates the housing.

5. The implantable medical device of claim 1, wherein the fibrous electrospun cover layer is permeable to one or more physiological chemical elements.

6. The implantable medical device of claim 1, wherein the fibrous electrospun cover layer is permeable to potassium and sodium.

7. The implantable medical device of claim 1, the fibrous electrospun cover layer comprising polyethylene glycol.

8. The implantable medical device of claim 16, the fibrous electrospun cover layer comprising a copolymer comprising polyethylene glycol subunits.

9. The implantable medical device of claim 8, the fibrous electrospun cover layer comprising polyethylene glycol molecules, wherein the polyethylene glycol molecules are covalently bonded to the thermoplastic fibers.

10. The implantable medical device of claim 1, the fibrous electrospun cover layer comprising a plurality of zones, wherein at least two of the plurality of zones have a different thickness, fiber density, fiber size, or fiber composition from one another.

11. The implantable medical device of claim 10, wherein at least one of the plurality of zones is disposed over the optical chemical sensing element and at least one of the plurality of zones is disposed over the housing.

12. The implantable medical device of claim 1, further comprising a frame, wherein the frame is disposed between the housing and the fibrous electrospun cover layer.

13. A method of making an implantable medical device comprising:
applying a fibrous electrospun cover layer over a frame member; and
fitting the frame member over at least one of a housing and a header of the implantable medical device.

14. The method of claim 13, wherein the fibrous electrospun cover layer is from 10 microns to 2 millimeters thick.

15. The method of claim 13, wherein the fibrous electrospun cover layer is permeable to one or more physiological chemical elements.

16. The method of claim 13, wherein the fibrous electrospun cover layer comprises a plurality of zones.

17. A method of making an implantable medical device comprising:
depositing an optical chemical sensing element into a sensor optical carrier attached to a housing; and
applying a fibrous electrospun cover layer over the optical chemical sensing element.

18. The method of claim 17, further comprising applying the fibrous electrospun cover layer over the optical chemical sensing element and the housing.

19. The method of claim 17, wherein the fibrous electrospun cover layer encapsulates the housing.

20. The method of claim 17, wherein applying a fibrous electrospun cover layer over the optical chemical sensing element further comprises a plurality of zones, wherein at least two of the plurality of zones have a different thickness, fiber density, fiber size, or fiber composition from one another, wherein at least one of the plurality of zones is disposed over the optical chemical sensing element and at least one of the plurality of zones is disposed over the housing.
